(19) United States
(12) Patent Application Publication
(10) Pub. No.: US 2014/0364786 A1
(43) Pub. Date: Dec. 11, 2014

(54) Universally adjustable lumbar brace

(71) Applicant: University Braces, LLC, San Diego, CA (US)

(72) Inventors: Thomas T. Haider, Rancho Santa Fe, CA (US); Gustavo R. Prado, San Diego, CA (US)

(73) Assignee: University Braces, LLC, San Diego, CA (US)

(21) Appl. No.: 13/999,029

(22) Filed: Jan. 4, 2014

Related U.S. Application Data
(60) Provisional application No. 61/832,658, filed on Jun. 7, 2013.

(51) Int. Cl. A61F 5/02

(52) U.S. Cl. CPC A61F 5/028
USPC 602/19

(57) ABSTRACT

A modular lumbar belt system has left and right belt sections arranged to be joined at their distal ends adjacent a patient's lumbar region with their proximal ends wrapped around his/her waist and secured together. The belt sections are formed with vertically oriented segments adapted to be selectively folded back against each other to adjust the length of the sections and thereby accommodate the waist sizes of a large population of patients. A conventional pulley-cord-tightening system allows the user to tighten the belt sections once the proximal ends are secured together. Modularity is achieved by providing 1) a lumbar support member which can be secured to the distal ends of the belt sections and detached when no longer needed and 2) lateral support members releasably secured to the belt sections about midway between the proximal and distal ends thereof.

### RELATED APPLICATION

This application is based on and claims priority of our U.S. provisional application No. 61832658, filed on Jun. 7, 2013, the contents of which are incorporated herein by reference.

### TECHNICAL FIELD

The present invention relates to a back brace employing a lumbar belt and pulley-cord system in particular to such a system which is universally adjustable to accommodate patients with different waist sizes, i.e., one “size fits all”. The invention eliminates the need for a multitude of different belt sizes, while providing ease of installation, and a comfortable fit enabling a patient to wear the brace in compliance with a physician's orders.

### BACKGROUND OF THE INVENTION

Lumbar braces have been used for years to immobilize the back of a patient and properly align the back after an injury as a result of trauma or disease. Some lumber braces may also reduce the load to intervertebral discs by increasing the patient's intracavitary pressure. With respect to the patented art, see U.S. Pat. No. 7,186,229 which discloses a corset-type arrangement with left and right belt sections and a pulley-cord system to tighten the corset against the user's waist. The system is not only complicated but many different sizes would be needed to accommodate different waist sizes.

The same brace or a close cousin is advertised by cybertechmedical.com, along with a product sizing table illustrating that some 7 different sizes are needed to accommodate a population of users, i.e., small (24″-30″), medium (30″-35″), large (35″-40″), etc. Also see Re: 35,940, US 2020/0204630, US 2011/0262056, U.S. Pat. No. 6,755,799, U.S. Pat. No. 7,346,558, U.S. Pat. No. 5,632,724, U.S. Pat. No. 5,776,087, US 2010/0262056, U.S. Pat. No. 5,346,461, U.S. Pat. No. 6,962,572, U.S. Pat. No. 7,727,172, and US 2009/0192425.

There is a need for a lumbar brace which is easy to install, user friendly and in particular adjustable in length, thereby eliminating the need for a multitude of different sizes of braces and modular in the sense that accessories needed initially may be removed when no longer needed.

### SUMMARY OF THE INVENTION

A universally adjustable lumbar brace, in accordance with the present invention, includes left and right belt sections, each section having a proximal and a distal end. The proximal ends are adapted to be wrapped around a patient's waist and secured together at the patient's front while the distal ends are connected together adjacent the patient's back via a pulley-cord-tightening system. The pulley-cord-tightening system (sometimes referred to herein as the “pulley-cord system”) allows the patient to grasp the cord(s), and with minimal effort pull the distal ends toward each other to tighten the belt sections around his/her waist, thus allowing for ease of manipulation even when the patient is suffering from debilitating and painful conditions. The reduction in the circumference of the belt causes an increase in the patient's intracavitiary pressure which reduces the load on the interverbral discs. The smaller intervertebral disc loads may promote an increase in disc height and a decrease of nervous tissue compression, leading to pain relief and an increase in comfort.

To achieve a “one size fits all” brace, each belt section is provided with sufficient free length, i.e., the length between the proximal and distal end when wrapped around a patient's waist, to accommodate a wide variety of waist sizes. An individual securing flap is affixed to the distal end of each belt section and extends forwardly toward the proximal end over a portion of the respective belt section. The anterior surfaces or faces of the securing flaps and posterior surfaces of or faces of the belt section having cooperating hook and loop fasteners to allow a selected portion of each belt section to be folded back over an adjacent portion and secured in place by the securing flap. The extended free belt length, in conjunction with the foldable flap feature and the securing flap, helps cut health care costs by (1) enabling the brace to accommodate the waist sizes of a large population of patients, greatly reducing the normally required inventory. and (2) avoiding the need for a different brace as the healing process occurs.

The pulley-cord-tightening system defines an imaginary vertical reference line with each belt section preferably defining a plurality of vertically oriented segments, i.e., extending parallel to the reference line. The segments are designed to selectively overlap each other when folded. Preferably the pulley-cord system comprises a pair of rigid-pulley-bearing plates with each plate mounted adjacent the distal end of a respective belt. A pair of cords, interleaved between the pulleys, are connected to a pair of pull handles in a conventional manner.

Preferably, the brace includes a back panel releasably connecting the distal ends of the belt sections together. In addition to the adjustability of the free length of the belt sections, the present invention provides a modular approach in the form of 1) a detachable semi-lumbar-sacral-posterior-rigid-support member (hereinafter “lumber-support member”) that provides sagittal-coronal support for the patient initially and can be removed when no longer needed and 2) removable semi-rigid-lateral panels that provide lateral support. These additional features take advantage of the conventional belt-tightening-pulley-cord system to conform to the patient's body, thereby increasing the brace's correctional support and comfort to the patient. Padding may also be used to minimize stresses to any soft tissue that comes in contact with the brace. The modularity of the brace is important because the patients and their health care providers can remove or add the modular components as the healing process progresses; this in turn increases the patient's compliance since bulk can be reduced as the patient needs less support.

### BRIEF DESCRIPTION OF THE DRAWNGS

FIG. 1 is an anterior perspective view of an assembled brace of the present invention including several attached modular features as wrapped around a patient (not shown);

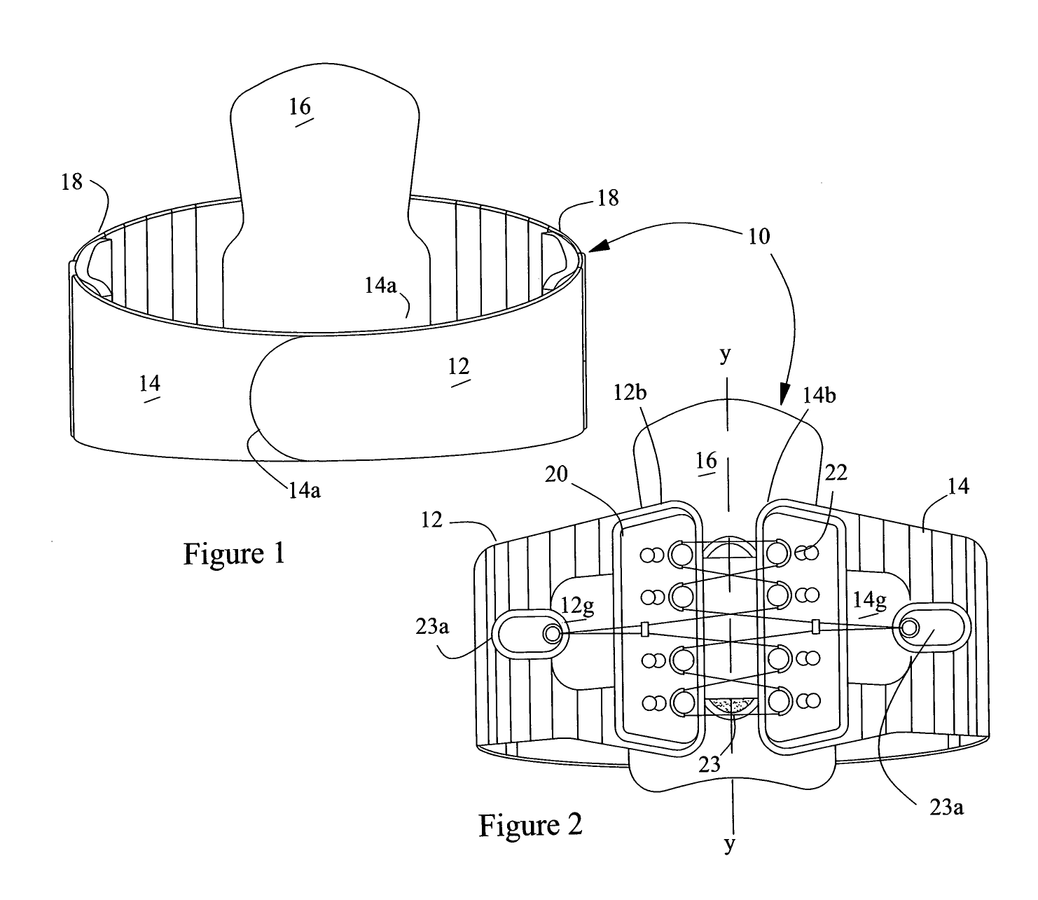

FIG. 2 is a posterior perspective view of the assembled brace of FIG. 1;

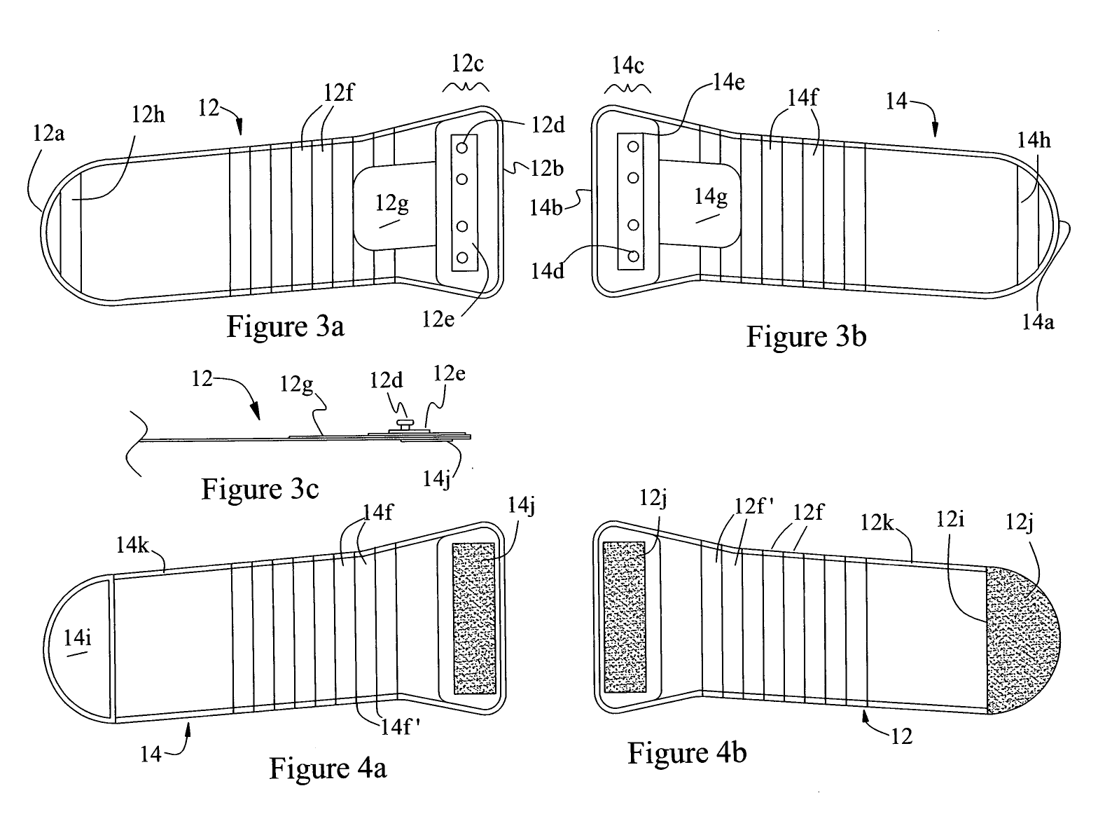

FIGS. 3a and 3b are posterior unassembled views of the left and right belt sections of the brace of FIG. 1;

FIG. 3c is a top view of a portion of the belt section of FIG. 3a;

FIGS. 4a and 4b are anterior views of the left and right belt sections;

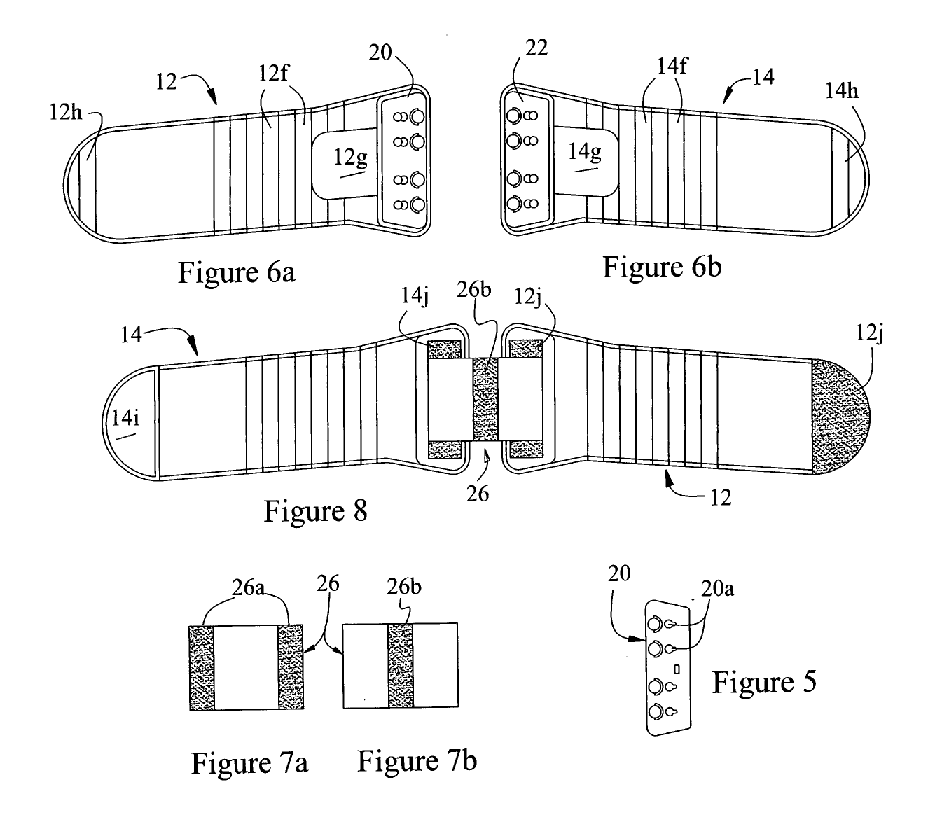

FIG. 5 is a posterior view of a pulley-carrying plate for the brace of FIG. 1;

FIGS. 6a and 6b are posterior views of the left and right belt sections assembled with pulley-carrying plates;

FIGS. 7a and 7b are posterior and anterior views, respectively, of a back panel for use to secure the distal ends of the belt sections together;

FIG. 8 is an anterior view of the left and right belt sections assembled to the back panel;

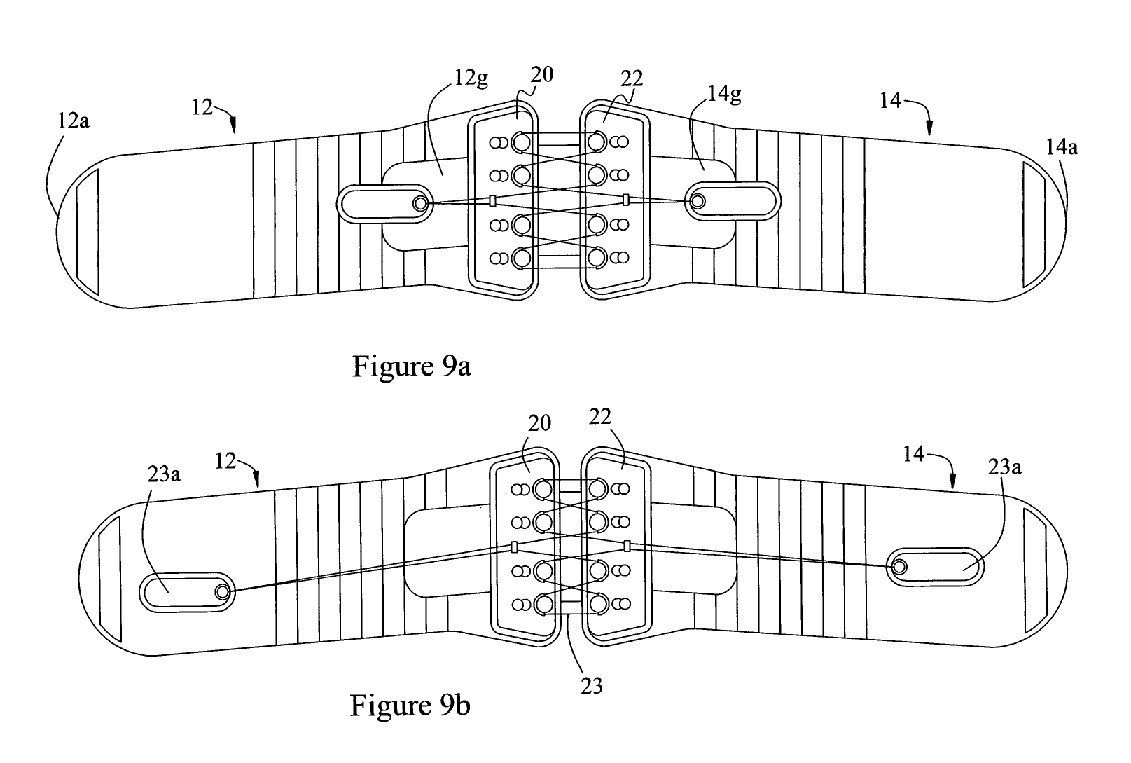

FIGS. 9a and 9b are posterior views of an assembled brace showing the pulley-cord system in a relaxed position (FIG. 9a) and in a tightened position (FIG. 9b);

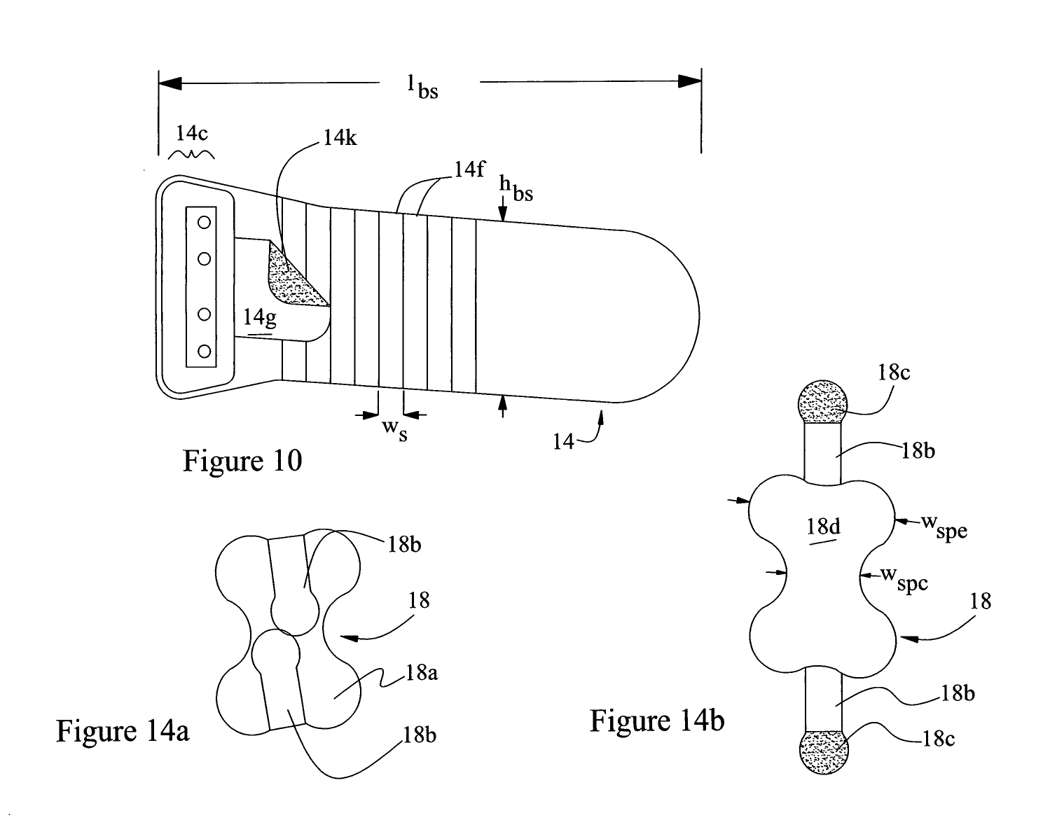

FIG. 10 is a somewhat enlarged view of a portion of the securing flap and underlying portion of the left belt section of FIG. 9b illustrating an arrangement for adjusting the free length of the belt sections;

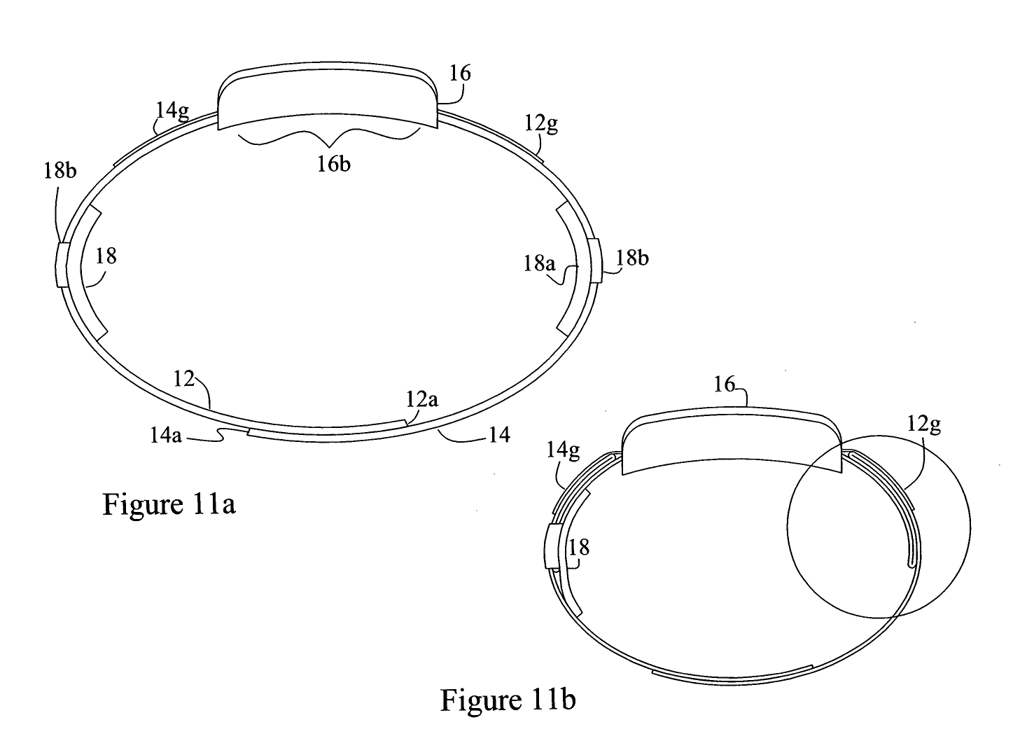

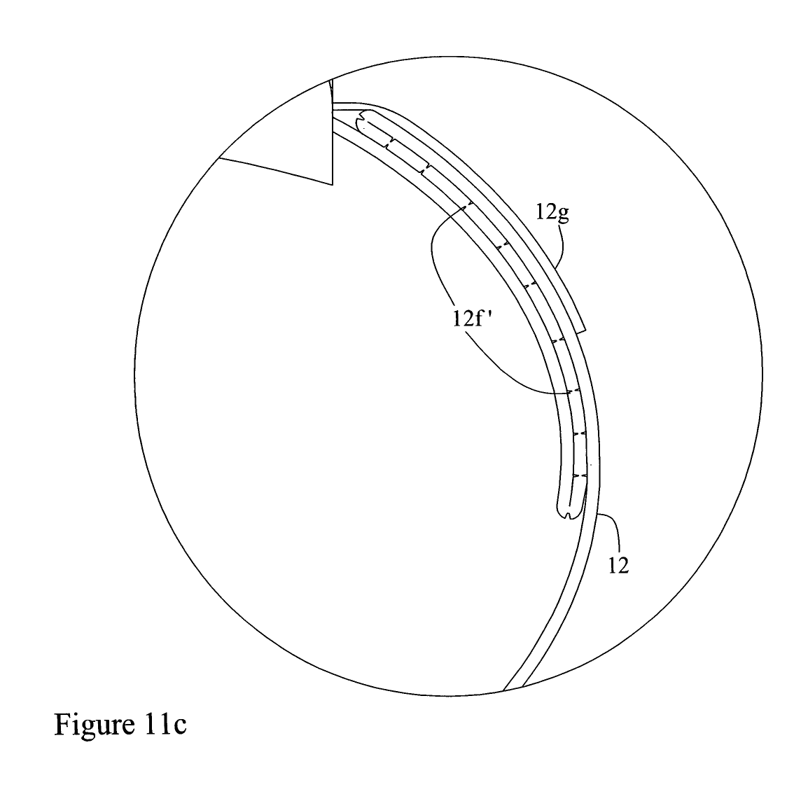

FIGS. 11a and 11b are assembled braces in a fully extended condition (FIG. 11a) and a fully retracted condition (FIG. 11b), as wrapped around a patient.

FIG. 11c is a blown up view of the circled portion of the left belt section of FIG. 11b showing the overlapping segments with the securing flap in place;

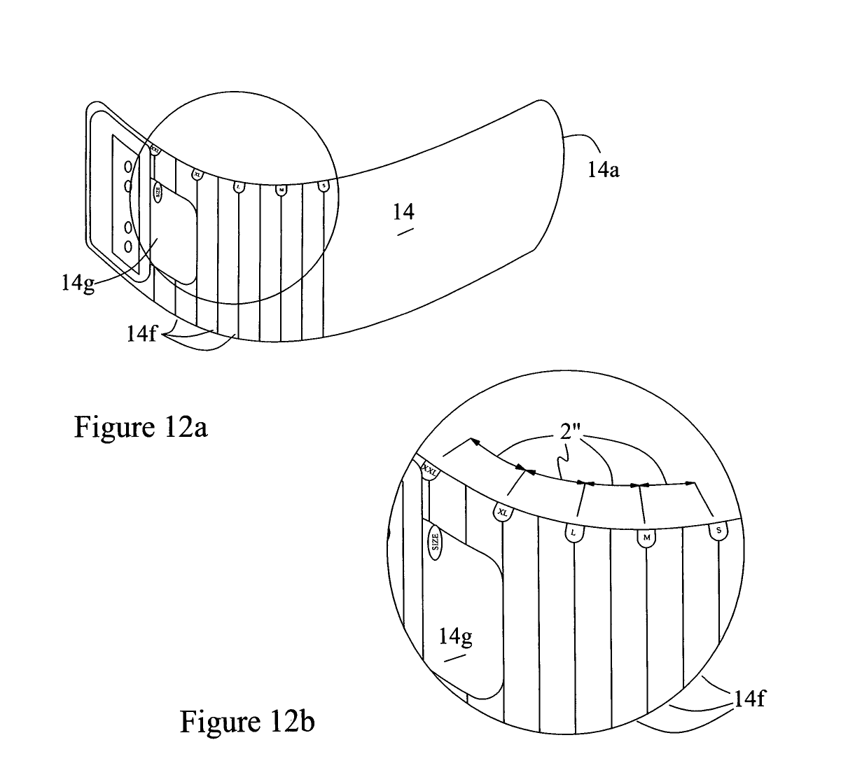

FIGS. 12a and 12b are a side view of the right belt section and an enlarged view of a portion of the section, respectively, illustrating how the folding procedure can reduce the free length of the section in specified increments;

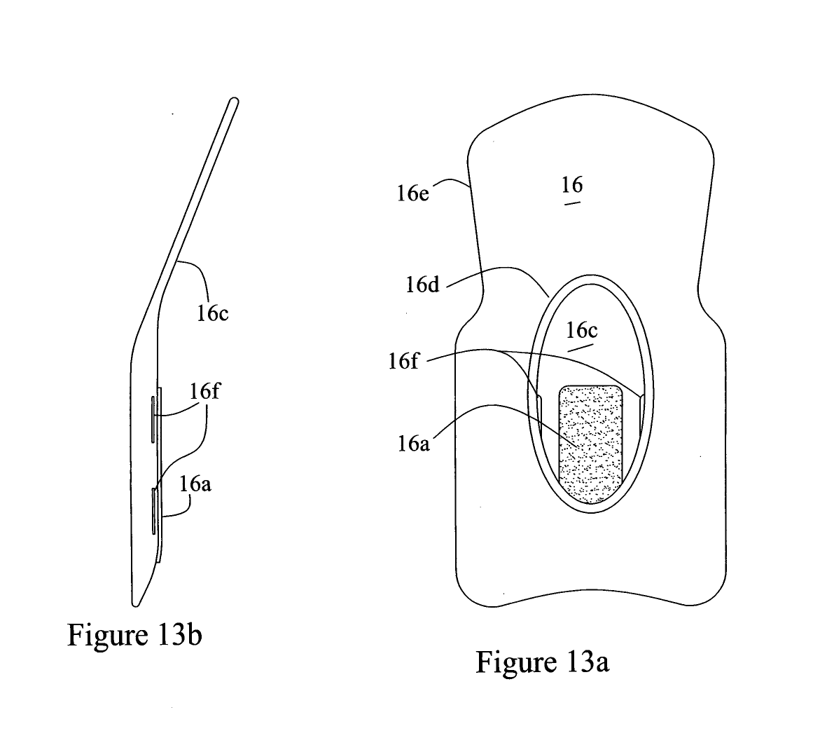

FIGS. 13a and 13b are posterior and side elevational views, respectively, of a detachable-lumber-support member which may be incorporated into the brace.

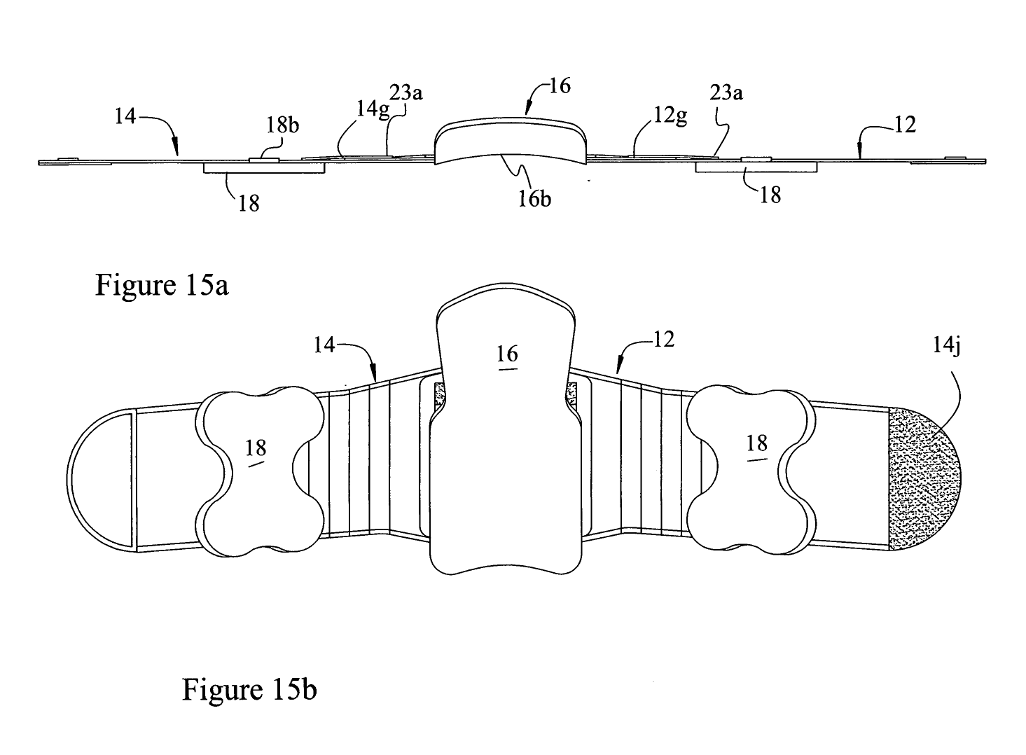

FIGS. 14a and 14b are posterior and anterior views, respectively, of lateral- support panels which may be incorporated into the brace, as illustrated in FIGS. 15a and 15b; and

FIGS. 15a and 15b are top and anterior views of the brace with the lumbar-support and lateral panels in place.

### DESCRIPTION OF THE PREFERRED EMBODIMENT

Referring now to FIGS. 1 and 2, the lumbar brace 10 of the present invention is shown as wrapped around a patient (not shown). The brace 10 includes left (12) and right (14) belt sections, a removable lumbar-posterior-support panel 16 and lateral support panels 18. Each belt section has a sufficient free length, i.e., distance between the proximal and distal ends prior to a folding procedure, to accommodate a very large waist size. A pulley-cord-tightening system comprising left (20) and right 22 pulley-cord-bearing plates is mounted adjacent the distal ends 12b and 14b, as shown. The pulley-cord system includes a conventional cord(s) 23 interleaved between the pulleys and terminating in a pair of pull handles 23a enabling a user to quickly grasp the handles to tighten the brace in a well known manner. The pulley-cord system defines an imaginary vertical-reference line y-y for purposes of defining the orientation of foldable-belt segments to be described. Sealing flaps 12g and 14g overlap several of the segments, as shown.

FIGS. 3a and 3b are posterior views of the left and right belt sections showing reinforced planar-pulley-receiving-rectangular portions 12c and 14c located adjacent the distal ends of the respective belt sections. Posts 12d and 14d extend outwardly from the rectangular portions 12c and 14e, as shown more particularly in FIG. 3c, a top view of the left belt section. Each belt section defines a plurality of vertically oriented, i.e., parallel to the reference line, foldable segments 12f and 14f. These segments can be folded back against an adjacent segment or segments to shorten the free length of a respective belt section, as will be explained. The posterior surfaces or faces of the belt sections, including the segments, are formed with Velcro-type loops to be engaged with cooperating hooks formed on the anterior surfaces of the securing flaps 12g and 14g. The flaps 12g and 14g extend forwardly from the pulley-receiving portions 12c and 14c and over the adjacent segment(s), as will be explained. Velcro is a trademark of Velcro USA, Inc. Straps 12h and 14h, located at the proximal end of the belt section, are provided for the convenience of a patient in putting the brace on.

Referring now to FIGS. 4a and 4b, and in particular surfaces 12j and 14j, these surfaces are formed with hook elements for engaging cooperating loops formed on posterior portions of a back panel, shown in FIGS. 7a and 7b. This panel secures the left and right belt sections together. The anterior side of the right belt section, at the proximal end thereof, forms a hand-receiving pocket 14i for the convenience of the patient in putting the brace on. The foldable segments are separated by vertically oriented stitching of the fabric forming the belt sections and designated by the reference numerals 12f′ and 14f in these figures as well as in FIG. 11c. This stitching between the segments form fold lines enabling the segments to be readily folded. The fold lines are not separately identified in the other figures to simplify the drawings. Peripheral hems 12k and 14k to form exterior borders are shown in these figures as well as in some of the other figures.

FIG. 5 illustrates a pulley-bearing plate 20 provided with openings 20a for receiving the posts 12d or 14d formed on portions 12c or 14c. The pulley-bearing plates 20, along with the cords 23, are off-the-shelf items and are available from Kao Chen Enterprises located in Taiwan. Republic of China.

FIGS. 6a and 6b show the separate belt sections with the pulley-bearing plates installed.

FIGS. 7a and 7b show a back panel for connecting the belt sections together. It is to be noted that the areas 26a (on the posterior side) are formed with loop fastener elements for cooperating with the hook elements 12j and 14j of the belt sections. The area 26b is also formed with loop fastener elements to engage the hook fastener elements on a removable lumbar-support panel to be discussed. It is to be noted that cross hatching the loop fastener elements is shown only for the back panel to preserve clarity in the drawings. The back panel , like the pulley-cord system, also defines the vertical reference line y-y.

FIG. 8 shows the anterior side of the belt sections assembled to the back panel 26.

FIGS. 9a and 9b show the belt sections received in a relaxed extended condition and in a tightening condition, respectively.

FIG. 10 is a posterior view of the right belt section with a corner 14k of the securing flap 14g folded back onto itself showing the hook fastening elements 14k formed on the underside of the securing flap. This arrangement, which is also present in the left-hand- belt section, allows the securing flaps to engage and secure the folded over segments 12f, 14f in place shortening the free length of the respective section. The labels_{lbs}, representing the free or fully extended condition of the belt may, for example only, be of the order of 26″. The label label_{hbs}, representing the nominal height of the central area of the belt section, may be about 6″, as an example only. The label_{ws}, representing the width of the individual segments may, for example, be between ½″ and 3″ and preferably about ¾″ to 1″.

FIGS. 11a and 11b show (in a top view) the assembled belt in an extended condition (to fit a very large waist) and in a retracted condition (to fit a small waist), respectively. The accessories items, i.e., the lumbar support 16 and the lateral supports 18 are also shown in place,

FIG. 11c shows, in an extended view, the overlapping arrangement of the individual segments 12f and the securing flap 12g.

FIGS. 12a and 12b illustrate, as an example, how individual segments measuring one inch each can be employed to provide 2 inch increments in the shortening of the belt sections, i.e., resulting in 8 total inches for each belt section.

FIGS. 13a and 13b illustrate a posterior side view and the removable lumbar-support panel member 16, respectively. The member has a lordotic curvature 16b in the lower portion to provide proper spine support and openings 16f to increase air circulation. See FIGS. 13b. The support member also includes an upwardly extending portion 16c. An area 16a containing hook fastener elements cooperates with the loop fastener elements 26b on the back panel to releasably secure the lumbar support to the brace, Reference 16d represents an opening in a cloth cover 16e to provide access to the fastening element 16a. The lumbar support member may be made of a semi-rigid material as ABS plastic and measure 15-20 inches long and 5-10 inches wide.

Referring now to FIGS. 14a and 14b a lateral support member 18 is arranged to be releasably attached to the inner side of each belt section at a desired location between the proximal and distal ends. The lateral supports 18 include an inner surface 18d, an outer surface 18a, and two support arms 18b, terminating in securing tabs 18c formed with hook fastener elements. The lateral support members are shown attached to the left and right belt sections in FIGS. 1, 11a and 15b.

FIGS. 15a and 16b show a top and an anterior view of the assembled brace including the lumbar support and lateral support members in place. The adjustable belt members may be made of nylon or polyester covering a PU foam filling. The lumber support may be made of nylon.

### CONCLUSION

There has been described a modular lumbar brace with detachable lumber and lateral support members and left and right belt sections which rely on foldable segments for length adjustment to accommodate a large population of patients while taking advantage of a conventional pulley-cord-tightening system to provide an easy to use “one size fits all” lumbar support system. Various modifications may occur to those skilled in the art without involving a departure from the spirit and scope of our invention as defined in the appended claims.

1. A universally adjustable lumbar brace comprising:
a) a back panel arranged go be placed against a patient's back and defming an imaginary vertical-reference line bisecting the back panel;
b) left and right side belt sections, each section having a proximal and a distal end, the proximal ends being arranged to be wrapped around a patient's waist and secured together, the distal ends being arranged to be secured to the back panel to allow the distal ends of the belt sections to be moved toward and away from each other;
c) each belt section being formed with a plurality of foldable segments extending parallel to the reference line, each segment being arranged to be releasably folded against the adjacent segments to adjust the length of the respective belt section forwardly of the distal end thereof to thereby accommodate different waist sizes;
d) a securing flap extending from each respective distal end over the adjacent foldable segments, the anterior surface of each securing flap and the posterior surface of each foldable segment having cooperating attaching means to allow the respective belt section to be shortened by folding one or more of the sections on top of each other and securing the top section to the securing flap; and
e) a conventional belt-tightening-pulley-cord section to allow the brace to be tightened once the appropriate length of the belt section has been decided upon and the proximal ends secured together.

2. The lumbar brace of claim 1 further including a semi-rigid-lumbar-posterior-support plate releasably secured to the back panel.

3. The lumbar support of claim 2 further including a semi-rigid-lateral-support panel releasably attached to each belt section intermediate the proximal and distal ends thereof;

4. A universally adjustable lumbar brace having left and right flexible belt sections with each belt section having a proximal and a distal end, the proximal ends being arranged to be wrapped around a patient's waist and secured together at the front of the patient, the distal ends being arranged to be joined together adjacent the back of the patient by a conventional pulley-cord-tightening system to allow the distance between the distal ends to be drawn toward each other to tighten the belt sections around the patient's waist, the improvement comprising:
a) the belt sections having sufficient free length between their proximal and distal ends to accommodate a variety of patient waist sizes;
b) individual securing flap affixed to the distal end of each belt section and extending over a portion of the respective belt section; and
b) the anterior faces of the securing flaps and the posterior faces of each of the belt sections having cooperating hook and loop fasteners to allow a selected portion of each belt section to be folded over and secured in place by the respective securing flap, thereby allowing a user or health care provider to adjust the free length of the respective belt for accommodating patients with different waist sizes.

5. The lumbar brace of claim 4 wherein the pulley-cord system defines an imaginary vertical-reference line and wherein each belt section defines a plurality of segments extending forwardly of the respective distal end and parallel to the reference line for facilitating the folding procedure of the belt sections.

6. The lumbar brace of claim 5 wherein the pulley-cord system is formed of left and right rigid-pulley-carrying plates each pulley-carrying plate secured to the distal end of a respective belt section and carrying a plurality of pulleys with a pair of cords interleaved between the pulleys and connected to a pair of pull handles and further including a back panel releasably securing the pulley-carrying panels together.

7. The lumbar brace of claim 6 further including a back panel for releasably securing the belt sections together.

8. The lumbar brace of claim 7 further including a rigid-lumber-posterior panel releasably secured to the back panel.

9. The lumbar brace of claim 8 further including a semi-rigid-side-support panel releasably secured to each belt section intermediate the proximal and distal ends.

10. An adjustable modular lumbar brace system comprising:
a) left and right belt sections, each section having a proximal and a distal end, the proximal ends adapted to be wrapped around a patient's waist and secured together at the patient's front, each belt section having a free length extending from the distal to the proximal;
b) left and right belt sections, each section having a proximal and a distal end, the proximal ends adapted to be wrapped around a patient's waist and secured together at the patient's front, each belt section having a free length extending from the distal to the proximal;
c) a conventional-pulley-cord-tightening system connected between the distal ends of the belt sections and defming an imaginary vertical reference line;
d) each belt section formed with a plurality of vertically oriented fold lines separating foldable segments, the segments having a posterior surface and being arranged to be folded back against adjacent segments to shorten the free length of the respective section;
e) a securing flap extending forwardly from the distal end of each respective belt section and over adjacent segments, the securing flaps having an anterior surface, the posterior and anterior surfaces of the securing flaps and the segments having cooperating hook and loop fasteners to allow the free length of each section to be adjusted by folding selected segments together securing the segments in their folded position by the securing flap, thereby accommodating a variety of waist sizes;
f) a semi-rigid-lumbar support member detachably connected to the distal ends of the belt sections for providing support for the patient's lumbar region as needed; and
g) a pair of semi-rigid-size support panels adapted to be detachably secured to the belt sections intermediate the proximal and distal ends.

11. The lumbar brace of claim 19 wherein the segments are between about ½″ and 3″ in width.

12. The lumbar brace of claim 11 wherein the segments are about ¾″ in width.

13. The lumbar brace of claim 11 wherein the nominal height of each belt section is about 6″.

14. The lumbar brace of claim 11 wherein each lateral support has a center and end width of about 3″ and 6″, respectively.

15. The lumbar brace of claim 11 wherein the free length of each belt section is about 26″.
